(19) United States
(12) Patent Application Publication
(10) Pub. No.: US 2008/0241949 A1
(43) Pub. Date: Oct. 2, 2008

(54) Process for preparing quetiapine fumarate

(76) Inventors: Vinod Kumar Kansal, Haryana (IN); Suhail Ahmad, New Delhi (IN); Kanhaiya Lal, Haryana (IN); Bhatu Tumba Patil, Maharashtra (IN)

Correspondence Address: KENYON & KENYON LLP, ONE BROADWAY, NEW YORK, NY, 10004, US

(21) Appl. No.: 12/080,140

(22) Filed: Mar. 31, 2008

Related U.S. Application Data
(60) Provisional application No. 60/920,936, filed on Mar. 29, 2007.
(60) Provisional application No. 60/920,963, filed on Mar. 29, 2007.

(51) Int. Cl. G01N 33/00; C07D 285/36

(52) U.S. Cl. 436/92; 540/547

(57) ABSTRACT

Provided is an improved synthesis of quetiapine and pharmaceutically acceptable salts.

### CROSS-REFERENCE TO RELATED APPLICATIONS

This application claims the benefit of U.S. provisional application Ser. No. 60/920,936 filed on Mar. 29, 2007 and U.S. provisional application Ser. No. 60/920,963, filed on March, 2007 filed hereby incorporated by reference.

### FIELD OF THE INVENTION

The present invention relates to improved synthesis of quetiapine and pharmaceutically acceptable salts.

### BACKGROUND OF THE INVENTION

Quetiapine, 2-(2-(4-dibenzo[b,f][1,4]thiazepin-11-yl-1-piperazinyl)ethoxy)ethanol, having the following chemical structure:

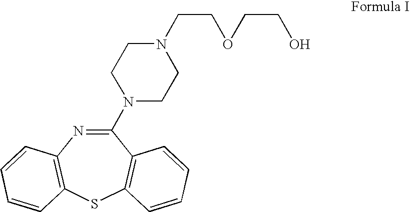

Quetiapine is a psychoactive organic compound that acts as an antagonist for multiple neurotransmitter receptors in the brain and acts as an antipsychotic agent reportedly useful for treating, among other things, schizophrenia. Merck Index, 13th Ed., 8130 (2001). This drug, having the CAS number: 111974-69-7, was approved under the trademark Seroquel®, by the U.S. Food and Drug Administration and is available from the innovator, AstraZeneca PLC. Quetiapine can be made, for example, as taught in the U.S. Pat. No. 4,879,288, (hereinafter “the '288 patent”) incorporated in its entirety herein by reference.

The '288 patent discloses preparing quetiapine by halogenating the compound of formula:

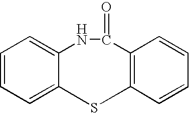

to obtain the compound of formula:

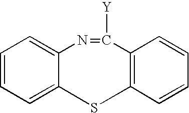

where Y can include a halogen. In example 1 of the '288 patent, a chlorination is carried out to obtain the compound of formula:

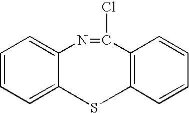

This chlorination reaction is carried out by combining the compound of formula:

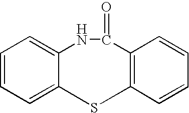

with phosphorous oxychloride and N,N-dimethylalanine to obtain a suspension. The suspension is then heated to reflux temperature. After 6 hours of heating, the resulting solution is allowed to cool. The excess phosphorus oxychloride is then removed using a rotary evaporator. The resulting product is then reacted with piperizine.

The '288 patent further discloses that: the “halogenating agent, [is] preferably a phosphorous pentahalide or oxyhalide (POHal₃). The above halide is selected, for example, from chlorine or bromine, especially chlorine. Where it is desired to . . . [chlorinate], a preferred halogenating agent is phosphorous oxychloride (POCl₃). Where it is desired to . . . [brominate], a preferred halogenating agent is phosphorous pentabromide. The reaction may advantageously be carried out in the presence of an N,N-disubstituted aniline, preferably N,N-di[1-6C]alkyl) substituted aniline, more preferably an N,N-dimethylaniline. The reaction is advantageously effected at an elevated temperature, preferably at the reflux temperature of the reaction mixture, conveniently for between 3 to 15 hours, preferably 4 to 10 hours, more preferably 6 hours.”

The scheme of the reaction provided in example 1 of the '288 patent is as follows:

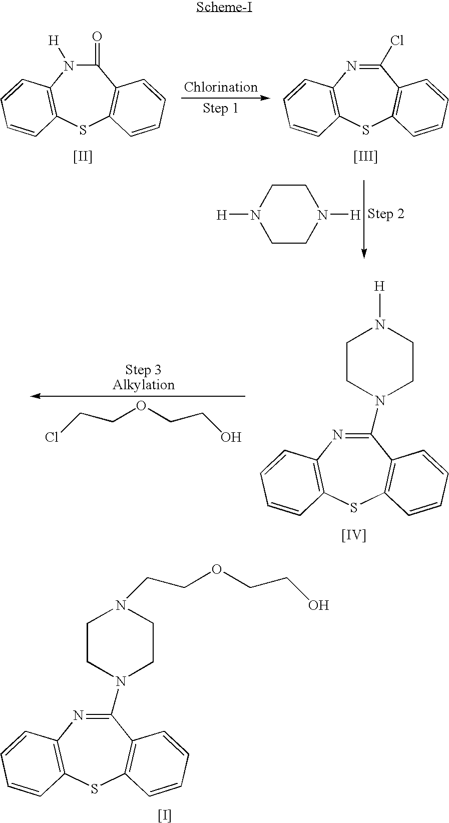

PCT patent publication WO 2006/135544 (hereinafter WO '544) carries out the same general reaction scheme but with different reagents. The abstract of WO '544 provides “a method for synthesizing 11-(4-[2-(2-hydroxyethoxy)ethyl]-piperazinyl)-dibenzo[b,f][1,4]thiazepine (quetiapine) and for recovering quetiapine as its fumarate salt in which dibenzo[b,f][1,4]thiazepine-11(10H)one is chlorinated in the presence of a trialkyl amine base using a slight molar excess of phosphorous oxychloride to produce 11-chloro-dibenzo[b,f][1,4]thiazepine which then is alkylated with piperazine to 11-piperazinyldibenzo[b,f][1,4]thiazepine, which finally is alkylated with 2-(2-chloroethoxy)ethanol.”

In example 1 of WO '544, chlorination is carried out in toluene by mixing compound of formula [III] with triethylamine and phosphorous oxychloride. The reaction is then carried out at the reflux temperature of 110° C. for two hours.

WO '544 prepares compound IV by reacting compound of formula [III] with piperizine. WO '544 reports that the reaction results in a dialkylated impurity of the following structure:

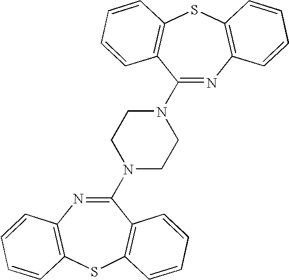

WO '544 reports removing this impurity by combining a toluene solution containing this impurity with aqueous HCl to obtain a pH of 3 in the aqueous phase. The addition of the acid results in formation of the salt of compound IV.

The processes of the '288 patent and WO '544 have certain drawbacks. The process of '288 patent as carried out in example 1 uses N,N-di[1-6C]alkyl) substituted aniline both as a base and a neat reagent. This compound is toxic. The process of the '288 patent as carried out in example 1 also uses large quantities of phosphorous oxychloride, which also is toxic and environmentally hazardous. Additionally, phosphorus oxychloride is typically removed via distillation in a cumbersome process.

The process of WO '544 replaces the N,N-di[1-6C]alkyl) substituted aniline of the '288 patent with a triethyl amine and toluene. The present Applicants have found that this process results in formation of additional impurities. Furthermore, triethylamine is extremely flammable. It is also corrosive and can cause burns. Chronic exposure to triethylamine may cause liver damage.

The present invention provides a process suitable for preparation of quetiapine with high purity on an industrial scale.

### SUMMARY OF THE INVENTION

In one embodiment, the present invention provides for a process for the preparation of the compound of formula [III], 11-halo-dibenzo[b,f][1,4]thiazepine said process comprising reacting the compound of formula [II], dibenzo[b,f][1,4]thiazepine 11-(10H) one with a slight excess of halogenating agent, wherein the process is carried out in the absence of a base. Preferably, the halogenating agent is a phosphorus pentahalide, oxyhalide (POHal₃), thionyl chloride or oxalylchloride. The reaction may advantageously be carried out in the presence of aliphatic halogenated hydrocarbon such as dichloromethane (MDC), ethylene dichloride (EDC) and the like at lower temperatures.

In another embodiment, the present invention relates to a method of preparation of 11-piperazinyldibenzo[b,f][1,4]thiazepine of formula [IV] and its pharmaceutically acceptable salts from 11-halodibenzo[b,f][1,4]thiazepine of formula [III] comprising: combining 11-halodibenzo[b,f][1,4]thiazepine of formula [III] with piperazine; adding an organic acid to obtain 11-piperazinyldibenzo[b,f][1,4]thiazepine of formula [IV]; and recovering compound of 11-piperazinyldibenzo[b,f][1,4]thiazepine of formula [IV] from the reaction mixture obtained. Preferably, the organic acid is an aliphatic organic acid which is selected from formic acid, acetic acid and adipic acid.

In yet another embodiment, the present invention provides an improved method of preparation of 11-piperazinyldibenzo[b,f][1,4]thiazepine of formula [IV] and its pharmaceutically acceptable salts from 11-halodibenzo[b,f][1,4]thiazepine of formula [III] comprising: combining 11-halodibenzo[b,f][1,4]thiazepine of formula [III] with piperazine to form a residue; crystallizing and/or slurrying the residue from C₁-C₅ alcohol to obtain the compound 11-piperazinyldibenzo[b,f][1,4]thiazepine of formula [IV]; and recovering.

In yet another embodiment, the present invention provides an improved method of preparation of 11-piperazinyldibenzo[b,f][1,4]thiazepine [IV] from 11-chloro-dibenzo[b,f][1,4]thiazepine [III] comprising: (a) reacting an aromatic solution of 11-chlorodibenzo[b,f][1,4]thiazepine with piperazine; (b) heating the solution; (c) cooling the solution to form a mixture having an aqueous and an organic layer; (d) separating the organic layer; (e) washing the organic layer with water; and (f) recovering the compound 11-piperazinyldibenzo[b,f][1,4]thiazepine of formula [IV] from the organic layer.

In one embodiment, the present invention encompasses a novel process for preparing Quetiapine fumarate, by preparing the compound of formula [IV] as described above, and converting it to Quetiapine and its pharmaceutically acceptable salts.

In one embodiment, the present invention provides an improved method of preparation of 2-(2-(4-dibenzo[b,f]-[1,4]thiazepine-11-yl-1-piperazinyl)ethoxy)ethanol of formula [I] from 11-piperazinyldibenzo[b,f][1,4]thiazepine of formula [IV] comprising: reacting solution of a compound of formula [IV] with 2-(2-chloroethoxy)ethanol in the presence of base, solvent and a phase transfer catalyst; heating; cooling; adding mineral acid or aliphatic organic acid to obtain the compound 2-(2-(4-dibenzo[b,f]-[1,4]thiazepine-11-yl-1-piperazinyl)ethoxy)ethanol of formula [I]; and recovering.

In another embodiment, the present invention provides isolated compounds having the following structure:

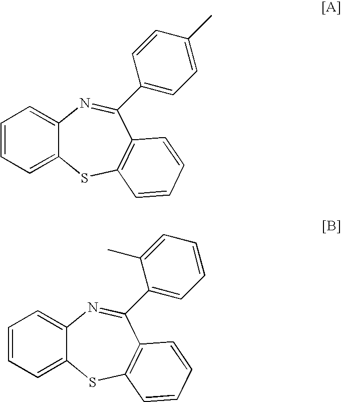

compound A and B, which are toluene related impurities are isolated during the preparation of the compound of the formula III from the compound of formula II.

In yet another embodiment, the present invention provides a novel process for preparing quetiapine fumarate, by preparing the compound of formula [I] (Quetiapine) as described above, and converting it to its pharmaceutically acceptable salts.

In one embodiment, the present invention provides an isolated compound of the following structure:

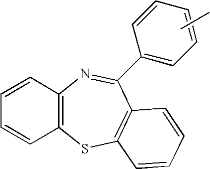

In another embodiment, the present invention provides a method for removing an impurity of the following structure:

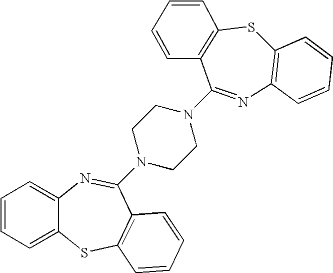

from 11-piperazinyldibenzo[b,f]thiazepine of formula [IV] comprising washing the 11-piperazinyldibenzo[b,f]thiazepine with an organic acid.

In yet another embodiment, the present invention provides a process for preparing quetiapine comprising
a) reacting a compound III of Formula

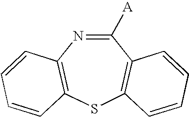

where A is chlorine, iodine or bromine, with piperizine to obtain a mixture of compound IV of formula:

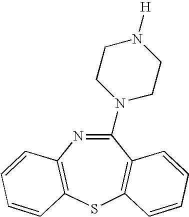

and an impurity of following structure:

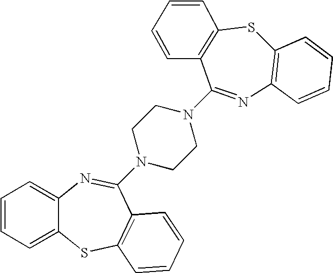

b) separating the impurity from compound of Formula IV by washing with an organic acid; and
c) converting compound of IV to quetiapine or a pharmaceutically acceptable salt.

In one embodiment, the present invention provides a process for preparing a compound III of the following structure:

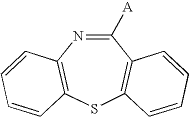

wherein A is chlorine, iodine or bromine comprising combining a compound II of the following structure:

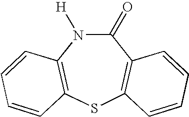

with a halogenating agent and an aliphatic halogenated hydrocarbon in the absence of a base to obtain compound III.

In another embodiment the present invention provides a process for preparing quetiapine comprising the steps of:
a) halogenting a compound II of formula:

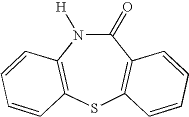

in the absence of a base by combining the compound II with an aliphatic halogenated hydrocarbon and a halogenating agent to obtain a compound III of formula:

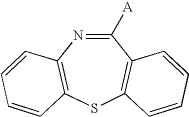

wherein A is chlorine, iodine or bromine;
b) reacting compound III with piperizine to obtain a compound IV of formula:

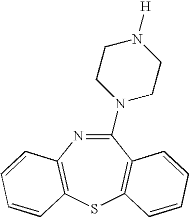

in a mixture with in impurity with following structure:

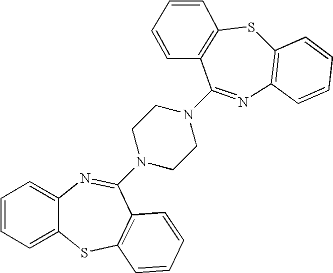

c) separating the from compound IV by at least one of combining the mixture with an organic acid or slurrying/crystallizing the mixture in a C₁-C₅ alcohol;
d) reacting compound IV with compound having the structure:

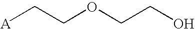

wherein A is chlorine, iodine or bromine, to obtain quetiapine of the following structure:

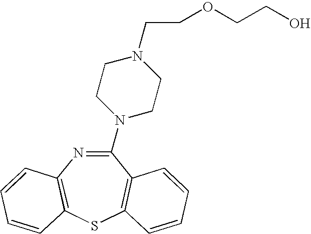

In another aspect of the present invention related to a improved method of preparation of 11-piperazinyldibenzo[b,f][1,4]thiazepine of formula [IV] and its pharmaceutically acceptable salts from 11-halodibenzo[b,f][1,4]thiazepine [III] comprising: combining 11-chlorodibenzo[b, f][1,4]thiazepine with piperazine to form a residue; crystallizing and/or slurrying the residue from C₁-C₅ alcohol to obtain the compound 11-piperazinyldibenzo[b,f][1,4]thiazepine of formula [IV]; and recovering the compound 11-piperazinyldibenzo[b,f][1,4]thiazepine of formula [IV], optionally, by reacting the aromatic solution of compound of formula [IV] with mineral halo acids converting it to Quetiapine and its pharmaceutically acceptable salts. Preferably, the mineral halo acid is HCl.

In another aspect, the present invention relates to improved method of preparation of 11-piperazinyldibenzo[b,f][1,4]thiazepine of formula [IV] from 11-chloro-dibenzo[b,f][1,4]thiazepine [III] comprising;
a) Reacting the aromatic solution of 11-chlorodibenzo[b,f][1,4]thiazepine with piperazine;
b) treating the residue with alcoholic solution; and
c) recovering the compound 11-piperazinyldibenzo[b,f][1,4]thiazepine of formula [IV].

In yet another aspect, the present invention relates to a improved method of preparation of 11-piperazinyldibenzo[b,f][1,4]thiazepine 2HCl from 11-halodibenzo[b,f][1,4]thiazepine [III] comprising;
a) reacting the aromatic solution of 11-halodibenzo[b,f][1,4]thiazepine of formula III with piperazine to obtain a reaction mixture;
b) acidifying the reaction mixture of step (a) with aliphatic organic acid, to form a reaction mixture
c) recovering compound 11-piperazinyldibenzo[b,f][1,4]thiazepine of formula (IV); from the reaction mixture of step (b) and reacting the aromatic solution of compound of formula (IV) with mineral hydrochloric acid to obtain the 11-piperazinyldibenzo[b,f][1,4]thiazepine 2HCl.

Preferably, the organic acid is an aliphatic organic acid which is selected from formic acid, acetic acid and adipic acid.

In step (a) the aromatic solution of 11-chlorodibenzo[b,f][1,4]thiazepine is combined with piperazine in step (b) of the process.

The solution is heated approximately between about 50° C. to about 110° C., preferably about 60° C. to about 80° C. and maintained about 1- to about 6 hrs, preferably about 2 to about 4 hrs.

The reaction mixture is cooled to about 20 to about 30° C. and filtered to isolate piperazinyl hydrochloride.

In the step (b) the mother liquor is washed with water and acidified using an aliphatic organic acid which is selected from formic acid, acetic acid and adipic acid; The pH of the solution is adjusted between about 5 to about 1, preferably about 4 to about 2 and most preferably between about 3.0 to about 2.0.

The phases then separated and the aqueous phase is extracted with organic solvent such as toluene. Thereafter 11-piperazinyl dibenzo[b,f][1,4]thiazepine compound was extracted from aqueous phase by adjusting pH between about 7.5 to about 11.0 preferably between about 8 to about 10 by using a suitable base selected from alkali metal carbonate, alkali metal hydroxide and alkali metal bicarbonate in the presence of organic solvent such as Methyl tert-butyl ether (MTBE), toluene, ethers, esters, chlorinated solvents and the like.

Compound of formula (IV) in the step (c) can be recovered and treated with HCl to get the compound of 2HCl salt of compound of formula [IV].

In yet another aspect, the present invention relates to an improved method of preparation of 11-piperazinyldibenzo[b,f][1,4]thiazepine 2HCl from 11-chloro-dibenzo[b,f][1,4]thiazepine [III] comprising;
a) Reacting the aromatic solution of 11-chlorodibenzo[b,f][1,4]thiazepine with piperazine;
b) treating the residue with alcoholic solution;
c) recovering the compound 11-piperazinyldibenzo[b,f][1,4]thiazepine of formula (IV); and
d) reacting the aromatic solution of compound of formula (IV) with hydrochloric acid.

### DETAILED DESCRIPTION OF THE INVENTION

As used herein, the term “room temperature” refers to a temperature of about 20° C. to about 30° C.

We have discovered that the process of WO '544 for preparation of iminohalide of formula [III] by using toluene and chlorinating agent such as POCl₃ under reflux conditions leads to the formation of toluene related impurities along with the desired imino halide of formula [III]. The toluene related impurities are namely compound of formula [A] and [B]

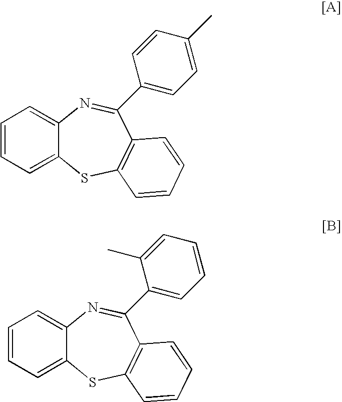

We have developed a new process to prepare compound III with a high purity by eliminating the base and using a halogenated hydrocarbon as a solvent. The use of the triethylamine of WO '544 would not be compatible with the process of the present invention since such bases are incompatible with halogenated hydrocarbons. The process of the present invention prepares a product with high purity by entirely eliminating the base of WO '544, and using a solvent that is incompatible with the process of WO '544.

The instant invention leads to the preparation of the 11-halodibenzo[b,f][1,4]thiazepine intermediate of formula [III] with a higher purity. It is also economically more suitable since it avoids an organic base and thus simplifies the overall procedure for scale-up, particularly the removal of toluene related impurities.

The present invention provides a process for preparing 2-(2-(4-dibenzo[b,f][1,4]thiazepin-11-yl-1-piperazinyl)ethoxy)ethanol of formula [I] starting with dibenzo[b,f][1,4]thiazepine 11-(10H) one of formula [II], as described in the following scheme:

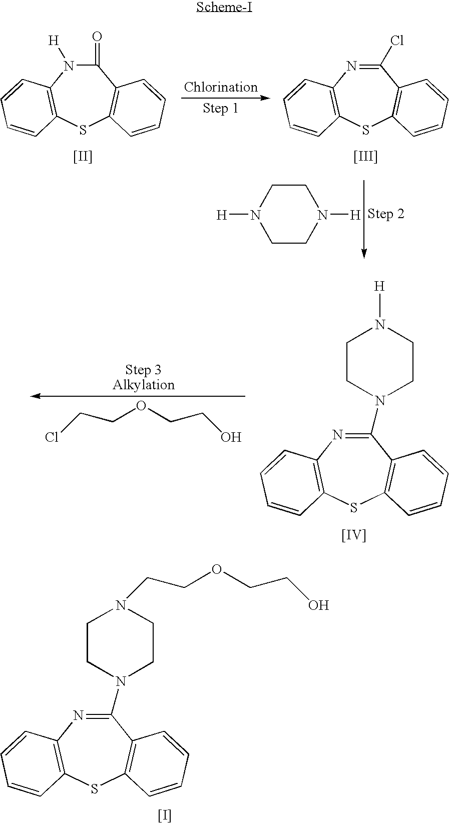

wherein A is a chlorine, iodine or bromine.

According to the present invention, the compound 11-halodibenzo[b,f][1,4]thiazepine of Formula [III] can be prepared by reacting the compound of formula [II] with a halogenating agent. For chlorination, examples of halogenating agents include phosphorus pentahalide (PCl₅), oxyhalide (POHal₃), thionyl chloride and oxalylchloride. Examples of brominating agents include phosphorus tribromide, bromine chloride, and aluminum tribromide. Preferably, chlorination is carried out. Preferably, a slight molar excess of the halogenating agent is used, such as about 1.2 to about 1.6. The reaction may be carried out in the presence of aliphatic halogenated hydrocarbon solvent such as a halogenated C₁-C₈ hydrocarbon. Examples of such hydrocarbons include dichloromethane (DCM) and ethylene dichloride (EDC). The temperature during the reaction is preferably about −5° C. to about −25° C., more preferably about −15° C. to about −20° C.

As exemplified, this reaction can be carried out by combining dibenzo [b,f][1,4]thiazepine-11(10H) one of formula [II] and a suitable solvent such as dichloromethane. A halogenating agent such as phosphorus pentachloride is then added. The addition of the halogenating agent is preferably done at below about room temperature, such as at about −25° C. to about −5° C., preferably about −20° C. to −15° C. After the addition of the halogenating agent, a reaction mixture is obtained. Preferably, the reaction mixture is maintained for about 120 to about 240 minutes, preferably about 120 to about 180 minutes. The reaction mixture may be further warmed, such as to about 20° C. to about 25° C. The reaction solvent can then be removed by evaporation such as by distillation. Toluene can be added and additional distillation carried out to remove additional dichloromethane. The reaction mixture can then be combined with water to obtain two layers. The product can be recovered from the organic layer by evaporation.

The halogenation reaction can be carried out in the absence of toxic and potentially carcinogenic amines, particularly N,N-disubstituted aniline, such as N,N-dimethylaniline. The halogenation reaction can also be carried out in the absence of a base.

The use of a halogenated hydrocarbon solvent in the synthesis allows for obtaining a product with a high level of purity, preferably higher than 95% as area percentage HPLC, more preferably about 99% HPLC purity. When toluene under reflux conditions is used instead, the product is impure, having a purity level of 87% by HPLC. Use of toluene leads to the formation of toluene related impurities along with the desired compound of formula [III]. The toluene related impurities have the following general structure:

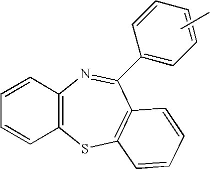

and specific compounds of the following structure:

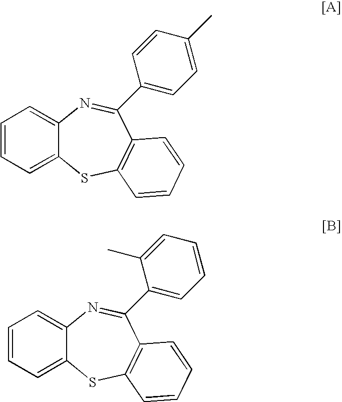

Also provided are these toluene related impurities in isolated form, substantially free of compound III (including where A is a chlorine). The isolation can be carried out by chromatography. Compounds A and B may form as separate distinct peaks. As used here, the term substantially free means that these compounds contain less than 1:1 molar ratio of compound of formula [III].

The above compounds A and B can be prepared by carrying the reaction described above but using toluene instead of a halogenated hydrocarbon solvent. The ideal conditions are under heating, preferably at reflux temperature. After a two phase reaction mixture is obtained as described above, a C₅-C₁₂ hydrocarbon such as n-hexane can be added to remove traces of toluene by distillation. Impurities A and B can be recovered as a residue by removal of the solvents. Impurity A and impurity B can be isolated from the residue using preparative TLC or chromatotron. The chromatotron may be: preparative, centrifugally, accelerated, radial, or thin-layer chromatograph.

The present invention further provides a process for converting the compound of formula [III] to compound of formula [IV] by with piperizine. The reaction can be carried out by combining 11-halodibenzo[b,f][1,4]thiazepine of formula [III] (preferably 11-chlorodibenzo[b,f][1,4]thiazepine) in a C₆-C₁₂ aromatic hydrocarbon such as toluene or xylene with piperazine. The reaction mixture can be heated approximately between about 50° C. to about 110° C., preferably about 60° C. to about 80° C. The reaction mixture can be maintained for about 1 to about 6 hours, preferably about 2 to about 4 hours.

The molar ratio of piperizine to compound of formula [III] (preferably 11-chlorodibenzo[b,f][1,4]thiazepine) can be about 3 to about 6. Preferably, the molar ratio is in a range of about 4 to about 5. Such excess molar ratio is preferred, so that HCl salt of piperazine is formed to neutralize the HCl formed during the reaction. This salt is formed as a soluble solid, which ultimately gets dissolved.

To recover the compound of formula [IV], the reaction mixture can then be cooled, such as to a temperature of about 20° C. to about 30° C. The cooling precipitates piperazinyl hydrochloride, which can be recovered by filtration. Water can then be added to the reaction mixture to obtain two layers. The organic layer is washed with water and acidified using an organic acid, preferably, C₁-C₈ aliphatic organic acid which is preferably formic acid acetic acid or adipic acid. The acidification precipitates out the compound of formula [IV]. Acidification can be carried out with formic acid, acetic acid and adipic acid. The pH is adjusted to about 5 to about 1, preferably about 4 to about 2 and most preferably between about 3 to about 2. Most preferably, the acidification is carried out at a pH of about 3.0. Thereafter 11-piperazinyl dibenzo[b,f][1,4]thiazepine of formula [IV] compound can be extracted from aqueous phase by adjusting pH between about 7.5 to about 11.0 preferably between about 8 to about 10 by using a suitable base selected from alkali metal carbonate, alkali metal hydroxide and alkali metal bicarbonate in the presence of organic solvent such as methyl tert-butyl ether (MTBE), toluene, ethers, esters, chlorinated solvents and the like.

The reaction mixture can be washed with water and the organic and aqueous layers are separated. The organic phase can be distilled off and toluene can be removed by adding a C₁-C₅ alcohol such as methanol, ethanol and n-butanol and crystallization and/or slurry with a C₁-C₅ alcohol such as methanol, ethanol and n-butanol to get the compound of formula [IV].

The reaction can be modified to obtain a hydrochloride salt (such as 2HCl salt) or another salt of compound of formula [IV]. After reaction in toluene, HCl or another acid can be added to the reaction mixture, such as at about room temperature. The reaction mixture can be heated, such as about to 105-110° C., and the water removed by evaporation, such as under azeotropic distillation. The HCl or another salt can then be recovered as a solid, such as by filtration. The resulting product can be slurried/crystallizing in a C₁-C₅ alcohol, such as absolute alcohol. The product can be dried, such as under a pressure of less than one atmosphere and a temperature of about 45-50° C.

In the reaction of compound of formula [III] (preferably 1′-chlorodibenzo[b,f][1,4]thiazepine) with piperizine, it is possible for the piperizine to react with two molecules of the compounds of formula [III], thus providing an impurity of the following structure (dialkylated piperizinyl):

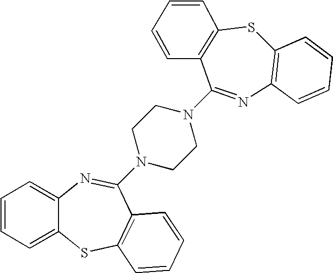

This impurity is not removed with aqueous washing and is believed to remain in the solution of 11-piperazinyldibenzo[b,f]thiazepine of formula [IV]. The impurity can be removed with washings with an organic acid, including aliphatic acids such as formic acid, acetic acid and adipic acid. The aliphatic acid is a C₁-C₈ acid. The washing can be carried out at lower temperature, such as about 20° C. to about 30° C., in the presence of the above mentioned aliphatic C₁ to C₈ acids. The removal of this impurity with aliphatic acid has advantages over removal with an aqueous acid. With a mineral acid, the pH may be widely and fall rapidly, but in case of organic acids like formic acid, acetic acid, even if the acid amount is on the high side, the pH range does not vary drastically. In case of mineral acid, the pH may go down and be unstable, while with the use of organic acids like formic acid, acetic acid, even if the acid amount is on the higher side, the pH range does not vary drastically. This results in a pH in a range of about 2-5, which in turn results in a good separation between the product and the impurity, ultimately giving a good yield. More preferably, the pH is in a range of about 2-4. The product of formula [IV] is obtained in the aqueous phase while the organic phase contains the dialkylated piperizinyl impurity. On the other hand, in the conversion of the compound of formula [IV] from compound of formula [III], when mineral acid is used as in WO 2006/135544, the dialkylated piperizinyl compound itself forms an acid salt, which is hard to remove. Compound IV can then be converted to compound I. This conversion can be carried out by reacting a solution of compound of formula (IV) with 2-(2-chloroethoxy)ethanol (or generally a 2-(2-halooethoxy)ethanol). Such reaction can be carried out by combining these compounds with a base, an organic solvent and optionally a phase transfer catalyst. To accelerate the reaction, the reaction mixture can be heated and subsequently cooled to facilitate recovery. After the reaction, water can added to obtain a two phases. An acid can be added to the aqueous phase to make the pH acidic. Once the acidic pH is achieved, compound IV in aqueous solution in salt form is obtained which is recovered by basification. Compound IV can then be recovered by evaporating any solvent, such as by azeotropic distillation.

Suitable phase transfer catalysts may be ammonium salts such as tricaprylylmethylammonium chloride (Aliquat® 336), tetra-n-butyl ammonium bromide (“TBAB”), benzyltriethylammonium chloride (“TEBA”), cetyltrimethylammonium bromide, cetylpyridinium bromide, N-benzylquininium chloride, tetra-n-butylammonium chloride, tetra-n-butylammonium hydroxide, tetra-n-butylammonium iodide, tetra-ethylammonium chloride, benzyltributylammonium bromide, benzyltriethylammonium bromide, hexadecyltriethylammonium chloride, tetramethylammonium chloride, hexadecyltrimethylammonium chloride, and octyltrimethylammonium chloride. More preferred phase transfer catalysts are Aliquat® 336, TBAB, TEBA and mixtures thereof, the most preferred being Aliquat® 336.

Suitable bases include alkali metal and alkaline earth metal carbonates or hydroxides, for example potassium bi/carbonate, sodium bi/carbonate, or sodium hydroxide, cesium carbonate/hydroxide. Metal carbonate is a preferred inorganic base for use in the practice of the present invention;

The reaction mixture can be heated at a temperature between about 60° C. to about 150° C., preferably about 80° C. to about 120° C.

Cooling can be done to a temperature of about 15° C. to about 30° C., preferably about 25° C. to about 30° C.

The acid can be a mineral acid such as HCl or H₂SO₄; or an organic acid such as formic acid, acetic acid or adipic acid;

The organic solvent may be selected from aromatic and aliphatic solvents. Aromatic solvents are selected from a group of toluene and xylene. Aliphatic solvents are selected from a group of aliphatic alcohols. Examples of aliphatic alcohols are C₁-C₈ alcohols like methanol, ethanol, n-butanol.

Compound I can be converted to a pharmaceutically acceptable salt such as a fumarate salt. Compound I obtained as described above can be combined with a C₁-C₄ alcohol, preferably absolute ethanol. Fumaric acid can then be added to obtain the fumarate, preferably at a temperature of about 40° C. to about 60° C. The fumarate can then be recovered by cooling, such as to about room temperature and by filtration. The wet material can dried under a pressure of less than one atmosphere and/or elevated temperature of about 40° C. to about 60° C. to afford quetiapine fumarate.

The isolated toluene related impurities may be used as reference markers/standards. A compound in a relatively pure state can be used as a “reference standard” (a “reference marker” is similar to a reference standard but it is used for qualitative analysis) to quantify the amount of the compound in an unknown mixture. When the compound is used as an “external standard,” a solution of a known concentration of the compound is analyzed by the same technique as the unknown mixture. (Strobel p. 924, Snyder p. 549) (Snyder, L. R.; Kirkland, J. J. Introduction to Modern Liquid Chromatography, 2nd ed. (John Wiley & Sons: New York 1979)). The amount of the compound in the mixture can be determined by comparing the magnitude of the detector response. See also U.S. Pat. No. 6,333,198, incorporated herein by reference.

The reference standard compound also can be used to quantify the amount of another compound in the mixture if the “response factor,” which compensates for differences in the sensitivity of the detector to the two compounds, has been predetermined. (Strobel p. 894). For this purpose, the reference standard compound may be added directly to the mixture, in which case it is called an “internal standard.” (Strobel p. 925, Snyder p. 552).

The reference standard compound can even be used as an internal standard when the unknown mixture contains some of the reference standard compound by using a technique called “standard addition,” wherein at least two samples are prepared by adding known and differing amounts of the internal standard. (Strobel pp. 391-393, Snyder pp. 571, 572). The proportion of detector response due to the reference standard compound that is originally in the mixture can be determined by extrapolation of a plot of detector response versus the amount of the reference standard compound that was added to each of the samples to zero. (e.g. Strobel, FIG. 11.4 p. 392).

A “reference marker” is used in qualitative analysis to identify components of a mixture based upon their position, e.g. in a chromatogram or on a Thin Layer Chromatography (TLC) plate (Strobel pp. 921, 922, 953). For this purpose, the compound does not necessarily have to be added to the mixture if it is present in the mixture. A “reference marker” is used only for qualitative analysis, while a reference standard may be used for quantitative or qualitative analysis, or both. Hence, a reference marker is a subset of a reference standard, and is included within the definition of a reference standard.

Although some of the knowledge of those in the art regarding reference standards has been described in general terms up to this point, those skilled in the art also understand that the detector response can be, for example, the peak heights or integrated peak areas of a chromatogram obtained, e.g. by UV or refractive index detection, from the eluent of an HPLC system or, e.g. flame ionization detection or thermal conductivity detection, from the eluent of a gas chromatograph, or other detector response, e.g. the UV absorbance, of spots on a fluorescent TLC plate. The position of the reference standard may be used to calculate the relative retention time for rosuvastatin and other impurities.

The above description of the present invention is illustrated in the form of working examples which are set forth in order to understand the present invention better and in no way limit the same in any aspect.

### Instruments:

1) pH meter; Toshniwal Research, pH/mV meter, Model: pH 100
2) HPLC Conditions: Chromatographic conditions for Impurity determination of Dibenzo[b,f][1.4]thiazepine of the formula II

### Chromatographic Conditions:

| Column | XTerra RP₈, 3.5μ, 150 × 4.6 mm, Waters | | |
|---|---|---|---|
| Flow | 1.5 ml/min | | |
| Sample volume | 10 μl | | |
| Detector | 250 nm | | |
| Column Temperature | 25° C. | | |
| Gradient Programming | Time | % Eluent A | % Eluent B |
| | 0.01 | 68 | 32 |
| | 5 | 45 | 55 |
| | 10 | 30 | 70 |
| | 15 | 20 | 80 |
| | 20 | 20 | 80 |
| | 25 | 45 | 55 |
| | 26 | 68 | 32 |
| | 30 | 68 | 32 |

### Buffer:

0.04M Ammonium acetate in water and 2.0 ml of 25% ammonium hydroxide per 1000 ml of buffer solution. Buffer pH should not be less than 9.2.

### Eluent A: Buffer

### Eluent B: Acetonitrile

### Diluent: Methanol

Before starting the analysis, washed the column for 30 min with Eluent A-20%: Eluent B-80%.

### Preparation of System Suitability Solution

Prepared about 1.0 mg/ml of DBT and 0.5 mg/ml of Quetiapine Fumarate in Methanol.

### Preparation of Sample Solution

Prepared accurately about 0.5 mg/ml solution of sample.

3) HPLC Conditions: Chromatographic conditions for Impurity determination of 11-chloro-dibenzo[b.f][1,4]thiazepine of the formula III

### Chromatographic Conditions

| Column | XTerra RP₈, 3.5μ, 150 × 4.6 mm, Waters | | |
|---|---|---|---|
| Flow | 1.5 ml/min | | |
| Sample volume | 10 μl | | |
| Detector | 250 nm | | |
| Column Temperature | 25° C. | | |
| Gradient Programming | Time | % Eluent A | % Eluent B |
| | 0.01 | 68 | 32 |
| | 5 | 45 | 55 |
| | 10 | 30 | 70 |
| | 15 | 20 | 80 |
| | 20 | 20 | 80 |
| | 25 | 45 | 55 |
| | 26 | 68 | 32 |
| | 30 | 68 | 32 |

Before starting the analysis, washed the column for 30 minutes with: Eluent A20%: Eluent B 80%

### Preparation of System Suitability Solution

Preparation about 1.0 mg/ml of Quetiapine Fumarate in Buffer: ACN(65:35v/v). (Solution A)

Prepared 0.2 mg/ml of DBT in methanol (Solution B).

Prepared 0.2 mg/ml of CDBT in diluent (Solution C).

Mix about 5 ml of Solution A and 2 ml each of Solution B and Solution C in a 10 ml volumetric flask. Makeup the volume with diluent.

### Preparation of Sample Solution

Prepared 0.5 mg/ml of sample in diluent.

4) HPLC Conditions: Chromatographic conditions for Impurity determination of Quetiapine Fumarate of the formula IV

### Chromatographic Conditions

| | |
|---|---|
| Column | XTerra RP₁₈, 5μ, 250 × 4.6 mm, Waters |
| Flow | 1.5 ml/min |
| Sample volume | 20 μl |
| Detector | 250 nm |
| Column Temperature | 45° C. |
| Diluent | Eluent A:Eluent B 60:40 |

Eluent A: 70% of ammonium acetate 0.04M in water, adjusted to pH 6.7 with either acetic acid or ammonia solution and 30% of Acetonitrile.

Eluent B: acetonitrile (gradient grade)

### Gradient Programming

| Time | % Eluent A | % Eluent B |
|---|---|---|
| 0 | 100 | 0 |
| 15 | 86 | 14 |
| 45 | 21 | 79 |

System suitability preparation: 2.5 mg of each DBTP-Thiazepine, dibenzo[b,f][1.4]thiazepine-11-(10H)-one and DBTP-ethyl in 100 ml of DMSO, and diluted in acetonitrile. Obtained concentration is of 0.0125 mg/ml. Weighed 5 mg of DBTP in 10 ml volumetric flask and added 1 ml of solution a) Dissolve with the diluent and bring to 10 ml with diluent.

5) HPLC Conditions: Chromatographic Conditions for Impurity determination of Quetiapine Fumarate of the Formula I

| | |
|---|---|
| Column: | XTerra RP₈, 3.5μ, 150 × 4.6 mm, Waters |
| Flow | 1.5 ml/min |
| Sample Volume | 20 μl |
| Detector | 250 nm |
| Column temperature | 45° C. |

### Preparation of Buffer

Prepare 0.04 M Ammonium acetate in water and add 2.0 ml of 25% ammonium hydroxide per 1000 ml of buffer solution. pH of the buffer should not be less than 9.2. Change the buffer daily.

### Eluent A: Buffer

Eluent B: Acetonitrile (gradient grade)

### Preparation of Diluent

### Eluent A: Eluent B (65:35) v/v

### Gradient Programming

| Time | % Eluent A | % Eluent B |
|---|---|---|
| 0 | 75 | 25 |
| 25 | 75 | 25 |
| 60 | 22 | 78 |

Equilibrium time: 8 minutes

Before starting an analysis, wash the column for 30 min with the following eluent Eluent A20%: Eluent B 80%. Mobile phase composition and flow rate may be varied in order to achieve the required system suitability.

### Preparation of System Suitability Solution

Prepare a mixture of about 1.0 mg/ml of Quetiapine fumarate standard and 0.002 mg/ml of DBTP standard in diluent.

### EXAMPLES

### Example 1

### 11-chlorodibenzo[b,f][1,4]thiazepine

A 1 liter round bottom flask equipped with stirring rod, thermo pocket, nitrogen inlet was charged with 50 gm (0.22 moles) of dibenzo[b,f][1,4]thiazepine-11-(10H)one, 300 cc dichloromethane and the mixture was stirred for 15 min. at room temperature. The resulting solution was chilled to −20° C. 70 gm (0.33 moles) phosphorus pentachloride was added in five equal lots at −20° C. to −15° C. over 90 min. The reaction mixture was maintained at −20° C. to −15° C. for 120-180 min. The reaction mixture was analyzed by HPLC. The analysis showed that less than 2% of dibenzo[b,f][1,4]thiazepine(10H)one was present. The reaction mixture was raised to 20° C. to 25° C. for 240 min., the reaction solvent (dichloromethane) was distilled off under vacuum below 40° C., leaving 50 cc dichloromethane with product. To the resulting reaction mass was added 250 cc toluene, the reaction solvent mixture (dichloromethane/toluene) was distilled off under vacuum below 55° C., leaving 150 cc toluene with product. 150 cc toluene was added, the reaction solvent was distilled off under vacuum below 55° C., leaving 150 cc toluene with product A further 250 cc toluene was added and the mixture cooled to room temperature. The reaction mixture was poured into 600 cc pre chilled DM water at 10-15° C., the resulting reaction mixture was stirred for 30 min at 25-30° C. The layers were separated and the organic layer washed with saturated brine solution. The organic layer was distilled off under vacuum below 55° C., leaving 350 cc toluene with product. Purity of 11-chlorodibenzo [b,f][1,4]-thiazepine in toluene was 99% (area % by HPLC).

### Example 2

### 11-chlorodibenzo[b,f][1,4]thiazepine

A 1 liter round bottom flask equipped with stirring rod, thermo pocket, nitrogen inlet was charged with 50 gm (0.22 moles) of dibenzo[b,f][1,4]thiazepine-11-(10H)one, 300 cc dichloromethane and the mixture was stirred for 15 min. at room temperature. The resulting solution was chilled to −20° C. 70 gm (0.33 moles) phosphorus pentachloride was added in five equal lots at −20° C. to −15° C. over 90 min. The reaction mixture was maintained at −20° C. to −15° C. for 120-180 min. The reaction mixture was analyzed by HPLC. The analysis showed that less than 2% of dibenzo[b,f][1,4]thiazepine(10H)one was present. The reaction mixture was raised to 20° C. to 25° C. for 30 min. The reaction mixture was poured into 600 cc pre chilled DM water at 10-15° C., the resulting reaction mixture was stirred for 30 min. at 25-30° C. The layers were separated and the organic layer washed with saturated brine solution. The organic layer was distilled off under vacuum below 50° C., leaving 50 cc dichloromethane with product. 150 cc toluene was added, the reaction solvent mixture (dichloromethane/toluene) was distilled off under vacuum below 55° C., leaving 100 cc toluene with product, to which was added 150 cc toluene, the reaction solvent was distilled off under vacuum below 55° C., leaving 100 cc toluene with product. To the resulting reaction mass was added 250 cc toluene and forwarded for the next step. Purity of 11-chlorodibenzo[b,f][1,4thiazepine in toluene was 99% (area % by HPLC).

### Example 3

### 11-chlorodibenzo[b,f][1,4]thiazepine

A 1 liter round bottom flask equipped with stirring rod, thermo pocket, nitrogen inlet, and water condenser was charged with 50 gm (0.22 moles) of dibenzo[b,f][1,4]thiazepine-1-(10H)one, 500 cc toluene and the mixture was stirred for 15 min. at room temperature. To the resulting solution was added 70 gm (0.33 moles) phosphorus pentachloride in five equal lots at 25° C. to 30° C. in 90 min. The reaction mixture was refluxed for 6 hrs at 110° C. The reaction mixture was analyzed by HPLC. The analysis result showed that less than 2% of dibenzo[b,f][1,4]thiazepine(10H)one was present. The reaction mixture was cooled to 20° C. to 25° C. for 30 min. The reaction mixture was poured into pre chilled DM water (500 cc) at 10-15° C., and was stirred for 30 min at 25-30° C. The layers were separated and the non aqueous layer washed with saturated brine solution. The non aqueous layer was distilled off under vacuum below 50° C. leaving 400 cc toluene with product and the resulting reaction mass forwarded for the next step. Purity of 11-chlorodibenzo[b,f][1,4]thiazepine in toluene was 87% (area % by HPLC).

### Example 4

### 11-piperazinyldibenzo[b,f][1,4]thiazepine

A 1 liter round bottom flask equipped with stirring rod, thermo pocket, water condenser was charged with solution of 11-chlorodibenzo[b,f][1,4]thiazepine in toluene 350 cc [52 gm (0.22 moles)], and was added 73.0 gm (0.84 moles) of piperazine at 45-50° C. The reaction mixture was heated to 70-80° C. The reaction mixture was maintained at 70° C. to 80° C. for 120-180 min. The reaction mixture was analyzed by HPLC. The reaction mixture was cooled to at 20° C. to 25° C. and was added 250 cc DM water and was stirred for 30 min. at 25-30° C. The layers were separated and the organic layer washed with 250 cc DM water. The organic layer was forwarded for the next step. Purity of 11-piperazinyldibenzo[b,f]-[1,4]-thiazepine in toluene was more than 97% (area % by HPLC).

### Example 5

### 11-piperazinyldibenzo[b,f][1,4]thiazepine

A 1 liter round bottom flask equipped with stirring rod, thermo pocket, water condenser was charged with solution of 11-chlorodibenzo[b,f][1,4]thiazepine in toluene [52 gm (0.22 moles)], and was added 73.0 gm (0.84 moles) of piperazine at 45-50° C. The reaction mixture was heated to 70-80° C. The reaction mixture was maintained at 70° C. to 80° C. for 120-180 min. The reaction mixture was analyzed by HPLC (to check for absence of compound of Formula III). The reaction mixture was cooled to at 20° C. to 25° C. was added 250 cc DM water and was stirred for 30 min. at 25-30° C. The layers were separated and the organic layer washed with 250 cc DM water. To the organic phase was added 250 cc water and was acidified with formic acid to obtain a pH of 2-3. The contents were stirred for 15 min. and the layers were separated. The aqueous layer was washed with 150 cc toluene and the aqueous layer was basified with sodium carbonate to a pH of 8 to 10 and extracted with 3×250 cc of toluene. Combine the organic layer and washed with DM water 130 cc twice. Purity of 11-piperazinyldibenzo[b,f][1,4]thiazepine in toluene was more than 99% (area % by HPLC).

### Example 6

### 11-piperazinyldibenzo[b,f]-[1,4]-thiazepine

A 1 liter round bottom flask equipped with stirring rod, thermo pocket, water condenser was charged with solution of 11-chlorodibenzo[b,f][1,4]-thiazepine in toluene [52 gm (0.22 moles)], and the mixture was stirred for 15 min 45-50° C. The resulting solution was added piperazine 73.0 gm (0.84 moles) at 45-50° C. The reaction mixture was heated to 70-80° C. The reaction mixture was maintained at 70° C. to 80° C. for 120-180 min. The reaction mixture was analyzed by HPLC (to check for absence of compound of Formula III). The reaction mixture was cooled to 20° C. to 25° C., to which, was added 250 cc DM water and was stirred for 30 min. at 25-30° C. The layers were separated and the organic layer washed with 250 cc DM water. To the organic phase was added 250 cc water and it was acidified with acetic acid to obtain a pH of 2-3. The contents were stirred for 15 min. and layers were separated. The aqueous layer was washed with 150 cc toluene and the aqueous layer was basified with sodium carbonate to a pH of 8 to 10 and extracted with 3×250 cc of toluene. Combine the organic layer and washed with DM water 130 cc twice. Purity of 11-piperazinyldibenzo[b,f]-[1,4]-thiazepine in toluene was more than 99% (area % by HPLC).

### Example 7

### 11-piperazinyldibenzo[b,f][1,4]thiazepine dihydrochloride

A 1 liter round bottom flask equipped with stirring rod, thermo pocket, water condenser was charged with solution of 11-piperazinyldibenzo[b,f][1,4]thiazepine in 700 cc toluene [63.0 g (0.22 moles)], and the mixture was stirred for 15 min 25-30° C. To the resulting solution was added conc. HCl 54 gm (0.53 moles) at 25-30° C. and the mixture was stirred for 15 min 25-30° C. The reaction mixture was heated to 105-110° C. and water was removed azeotropically. The resulting reaction mass was cooled to 25-30° C., and maintained for 2 hrs at 25-30° C. The hydrochloride salt was filtered under nitrogen atmosphere and washed with 50 cc toluene. The wet hydrochloride salt was slurry washed with abs. ethanol. The suck dried wet cake was dried under vacuum at 45-50° C. for 10 hrs. Dry weight of the hydrochloride salt was 70-75 gm. Purity of 11-piperazinyldibenzo[b,f][1,4]thiazepine dihydrochloride was more than 99.0% (area % by HPLC).

### Example 8

### 1-piperazinyldibenzo[b,f][1,4]thiazepine

A 1 liter round bottom flask equipped with stirring rod, thermo pocket, water condenser was charged with solution of 11-chlorodibenzo[b,f][1,4]thiazepine in toluene [52 gm (0.22 moles)], and the mixture was stirred for 15 min 45-50° C. To the resulting solution was added piperazine 73.0 (0.84 moles) at 45-50° C. The reaction mixture was heated to 70-80° C. The reaction mixture was maintained at 70° C. to 80° C. for 120-180 min. The reaction mixture was analyzed by HPLC (to check for absence of compound of formula III) and was cooled to 20° C. to 25° C. The reaction mixture was added 250 cc DM water and was stirred for 30 min. at 25-30° C. The layers were separated and the organic layer washed with 250 cc DM water. The organic phase was distilled off under vacuum below 70° C. Traces of toluene were removed by adding n-butanol. To the resultant oily mass was added 150 cc n-butanol. The mixture was stirred for 24 hrs and chilled to 0-5° C. The reaction mass was filtered with the filtrate (mother liquor) containing 11-piperazinyldibenzo[b,f][1,4]thiazepine. Purity of 11-piperazinyldibenzo[b,f][1,4]thiazepine in toluene was more than 98.0% (area % by HPLC).

### Example 9

### 11piperazinyldibenzo[b,f][1,4]thiazepine

A 1 liter round bottom flask equipped with stirring rod, thermo pocket, water condenser was charged with solution of 11-chlorodibenzo[b,f][1,4]thiazepine in toluene [52 gm (0.22 moles)], and the mixture was stirred for 15 min 45-50° C. To the resulting solution was added piperazine 73.0 gm (0.84 moles) at 45-50° C. The reaction mixture was heated to 70-80° C. The reaction mixture was maintained at 70° C. to 80° C. for 120-180 min. The reaction mixture was analyzed by HPLC and was cooled to 20° C. to 25° C. To the reaction mixture was added 250 cc DM water and was stirred for 30 min. at 25-30° C. The layers were separated and the organic layer washed with 250 cc DM water. The organic phase was distilled-off under vacuum below 70° C. The traces of toluene were removed by adding abs. ethanol. To the resultant oily mass was added 150 cc abs. ethanol. The mixture was stirred for 24 hrs and chilled to 0-5° C. The reaction mass was filtered with the filtrate ML (mother liquor) containing 11-piperazinyldibenzo[b,f][1,4]thiazepine. Purity of 11-piperazinyldibenzo[b,f][1,4]thiazepine in toluene was more than 98.0% (area % by HPLC).

### Example 10

### 2-(2-(4-dibenzo[b,f][1,4]thiazepine-11-yl-1-piperazinyl)ethoxy)ethanol OR 11-[4-[2-(2-hydroxyethoxy)ethyl]-1-piperazinyl]dibenzo[b,f][1,4]thiazepine

A 1 liter round bottom flask equipped with stirring rod, thermo pocket, water condenser was charged with solution of 11-piperazinyldibenzo[b,f][1,4]thiazepine in toluene 350 cc [63.0 g (0.22 moles)] and the mixture was stirred for 15 min 25-30° C., and, to which, was added sodium carbonate [41.0 gm (0.39 moles)], tetra butyl ammonium bromide [16.0 gm (0.05 moles)] and 2-(2-chloroethoxy)ethanol [32.0 gm (0.257 moles)] at room temperature. The reaction mixture was heated to reflux at 110-112° C. The reaction mixture was maintained at reflux for 10-12 hrs. The reaction mixture was analyzed by HPLC (to check absence of compound of formula IV) and was cooled to 25° C. to 30° C. To which, was added 150 cc DM water. Then the reaction mixture was stirred for 30 min at 25-30° C. The layers were separated and the aqueous layer extracted with 50 cc toluene. The extracts and the organic layer were combined, and the pH was adjusted to 2-3 using 1N HCl solution in DM (demineralized) water, the reaction mixture was then stirred for 30 min at 25-30° C. The layers were separated and the aqueous layer washed with 100 cc toluene twice. To the aqueous layer was added 250 cc toluene, and the pH was adjusted to 8-10 using sodium carbonate, the reaction mixture was stirred for 30 min at 25-30° C. The layers were separated and the aqueous layer extracted with 125 cc toluene. The extracts and the organic layer were combined, and washed with DM (demineralized) water 300 cc twice. The organic layer was distilled-off under vacuum below 70° C. to afford 2-(2-(4-dibenzo[b,f]-[1,4]thiazepine-11-yl-1-piperazinyl)ethoxy)ethanol Purity of 2-(2-(4-dibenzo[b,f]-[1,4]thiazepine-11-yl-1-piperazinyl)ethoxy)ethanol was 99.0 (area % by HPLC).

### Example 11

### 2-(2-(4-dibenzo[b,f][1,4]thiazepine-11-yl-1-piperazinyl)ethoxy)ethanol OR 11-[4-[2-(2-hydroxyethoxy)ethyl]-1-piperazinyl]dibenzo[b,f][1,4]thiazepine

A 1 liter round bottom flask equipped with stirring rod, thermo pocket, water condenser was charged with solution of 11-piperazinyldibenzo[b,f][1,4]thiazepine in toluene 350 cc [63.0 g (0.22 moles)] and the mixture was stirred for 15 min at 25-30° C. Sodium carbonate [41.0 gm (0.39 moles)], tetra butyl ammonium bromide [16.0 gm (0.05 mole)] and 2-(2-chloroethoxy)ethanol [32.0 gm (0.257 moles)] were added at room temperature. The reaction mixture was heated to reflux at 110-112° C. The reaction mixture was maintained at reflux for 10-12 hrs. The reaction mixture was analyzed by HPLC (to check the absence of compound of Formula IV) and was cooled to 25° C. to 30° C., and was added 150 cc DM water. The reaction mixture was then stirred for 30 min at 25-30° C. The layers were separated and the aqueous layer extracted with 50 cc toluene. The extract and the organic layer were combined, to which was added 250 cc water and was acidified with formic acid to obtain a pH of 2-3. The reaction mixture was stirred for 30 min at 25-30° C. The layers were separated and the aqueous layer washed with 100 cc toluene twice. To the aqueous layer was added 250 cc toluene, and the pH adjusted to 8-10 using sodium carbonate. The resulting reaction mixture was stirred for 30 min at 25-30° C. The layers were separated and the aqueous layer extracted with 125 cc toluene. The organic layers were combined and washed with DM water 300 cc twice. The organic layer was distilled off under vacuum below 70° C. to afford 2-(2-(4-dibenzo[b,f]-[1,4]thiazepine-11-yl-1-piperazinyl)ethoxy)ethanol. Purity of 2-(2-(4-dibenzo[b,f]-[1,4]thiazepine-11-yl-1-piperazinyl)ethoxy)ethanol was 99.0% (area % by HPLC).

### Example 12

### 2-(2-(4-dibenzo[b,f][1,4]thiazepine-11-yl-1-piperazinyl)ethoxy)ethanol OR 11-[4-[2-(2-hydroxyethoxy)ethyl]-1-piperazinyl]dibenzo[b,f][1,4]thiazepine

A 1 liter round bottom flask equipped with stirring rod, thermo pocket, water condenser was charged with solution of 11-piperazinyldibenzo[b,f][1,4]thiazepine in toluene 350 cc [63.0 g (0.22 moles)] and the mixture was stirred for 15 min 25-30° C., and was added sodium carbonate [41.0 gm (0.39 moles)], tetra butyl ammonium bromide [16.0 gm (0.05 mole)] and 2-(2-chloroethoxy)ethanol [32.0 gm (0.257 moles)] at room temperature. The reaction mixture was heated to reflux at 110-112° C. The reaction mixture was maintained at reflux for 10-12 hrs. The reaction mixture was analyzed by HPLC (to check for absence of compound of Formula IV) and was cooled to 25° C. to 30° C. To which, was added 150 cc DM water, then the reaction mixture was stirred for 30 min at 25-30° C. The layers were separated and the aqueous layer extracted with 50 cc toluene. The extract and the organic layer were combined, to which was added 250 cc water and was acidified with acetic acid to obtain a pH of 2-3. The reaction mixture was stirred for 30 min at 25-30° C. The layers were separated and the aqueous layer washed with 100 cc toluene twice. To the aqueous layer was added 250 cc toluene, and the pH was adjusted to 8-10 using sodium carbonate, the reaction mixture was stirred for 30 min at 25-30° C. The layers were separated and the aqueous layer extracted with 125 cc toluene. The extract and the organic layer were combined, to which was washed with DM (dimineralized) water 300 cc twice. The organic layer was distilled off under vacuum below 70° C. to afford 2-(2-(4-dibenzo[b,f]-[1,4]thiazepine-11-yl-1-piperazinyl)ethoxy)ethanol. Purity of 2-(2-(4-dibenzo[b,f]-[1,4]thiazepine-11-yl-1-piperazinyl)ethoxy)ethanol was 99.0% (area % by HPLC).

### Example 13

### 2-(2-(4-dibenzo[b,f][1,4]thiazepine-11-yl-1-piperazinyl)ethoxy)ethanol salt of fumaric acid OR 11-[4-[2-(2-hydroxyethoxy)ethyl]-1-piperazinyl]dibenzo[b,f][1,4]thiazepine fumarate

A 1 liter round bottom flask equipped with stirring rod, thermo pocket, water condenser was charged with solution of 11-piperazinyldibenzo[b,f][1,4]thiazepine in toluene 350 cc [63.0 g (0.22 moles)] and the mixture was stirred for 15 min 25-30° C., to which, was added sodium carbonate [41.0 gm (0.39 moles)], tetra butyl ammonium bromide [16.0 gm (0.05 mole)] and 2-(2-chloroethoxy)ethanol [32.0 gm (0.257 moles)] at room temperature. The reaction mixture was heated to reflux at 110-112° C. The reaction mixture was maintained at reflux for 10-12 hrs. The reaction mixture was analyzed by HPLC (to check for absence of compound IV) and was cooled to at 25° C. to 30° C., and was added 150 cc DM water, the reaction mixture was stirred for 30 min at 25-30° C. The layers were separated and the aqueous layer extracted with 50 cc toluene. The extract and the organic layer were combined, and the pH was adjusted to 2-3 using 1N HCl solution in DM water, the reaction mixture was stirred for 30 min at 25-30° C. The layers were separated and the aqueous layer washed with 100 cc toluene twice. To the aqueous layer was added 250 cc toluene, and the pH was adjusted to 8-10 using sodium carbonate, the reaction mixture was stirred for 30 min at 25-30° C. The layers were separated and the aqueous layer extract with 125 cc toluene. The extract and the organic layer were combined, and washed with DM water 300 cc twice. The organic layer was distilled off under vacuum below 50° C. leaving 50-60 cc toluene with product. Purity of 2-(2-(4-dibenzo[b,f]-[1,4]thiazepine-11-yl-1-piperazinyl)ethoxy)ethanol in toluene was 99.0% (area % by HPLC). To this solution 1000 cc absolute ethanol was added with activated carbon 5.0 gm and heated to reflux for 90 min. The resulting solution was cooled to 50-55° C. and filtered. The resulting solution was added 12.5 gm (0.5 moles) fumaric acid at 50° C. The reaction mixture was heated to reflux for 2 hrs and was slowly cooled to room temperature and maintained for 2 hrs at room temperature. The reaction mass was filtered and washed with 200 cc absolute ethanol. The wet material obtained was dried under vacuum at 50-55° C. to afford quetiapine fumarate. Dry weight of quetiapine fumarate is 60-65 gm. Purity of quetiapine fumarate was 99.5% (area % by HPLC).

### Example 14

### 2-(2-(4-dibenzo[b,f][1,4]thiazepine-11-yl-1-piperazinyl)ethoxy)ethanol salt of fumaric acid OR 11-[4-[2-(2-hydroxyethoxy)ethyl]-1-piperazinyl]dibenzo[b,f][1,4]thiazepine fumarate

A 1 liter round bottom flask equipped with stirring rod, thermo pocket, water condenser was charged 75 gm (0.2 moles) 2-(2-(4-dibenzo[b,f]-[1,4]thiazepine-11-yl-1-piperazinyl)ethoxy)ethanol, to which, was added 300 cc n-butanol. The mixture was heated to a clear solution and activated carbon 7.5 gm was added and heated to 60-70° C. The reaction mass was maintained for 90 min at 60-70° C. The reaction mass was filtered and washed with 75 ml n-butanol. To the filtrate ML (mother liquor) was added a fumaric acid solution at 50-60° C. [11.5 gm (0.1 moles) fumaric acid dissolved in 300 cc n-butanol at 80-85° C.]. The reaction mass was maintained at 50-60° C. for 30-60 min. and was slowly cooled to room temperature and maintained for 2 hrs at room temperature. The reaction mass was filtered and washed with 75 cc n-butanol & recrystallization form 1275 cc absolute ethanol. The wet material obtained was dried under vacuum at 50-55° C. to afford quetiapine fumarate. Dry weight of quetiapine fumarate is 60-65 gm. Purity of quetiapine fumarate was more than 99.5% (area % by HPLC).

### Example 15

### 2-(2-(4-dibenzo[b,f][1,4]thiazepine-11-yl-1-piperazinyl)ethoxy)ethanol salt of fumaric acid OR 11-[4-[2-(2-hydroxyethoxy)ethyl]-1-piperazinyl]dibenzo[b,f][1,4]thiazepine fumarate

A 1 liter round bottom flask equipped with stirring rod, thermo pocket, water condenser was charged with solution of 11-piperazinyldibenzo[b,f][1,4]thiazepine dihydrocholride [81.0 gm (0.22 moles)], in n-butanol 450 cc and the mixture was stirred for 15 min 25-30° C. To the resulting solution was added sodium carbonate [46.0 gm (0.44 moles)], tetra butyl ammonium bromide [16.0 gm (0.05 mole)] and 2-(2-chloroethoxy)ethanol [32.0 gm (0.257 moles)] at room temperature. The reaction mixture was heated to reflux at 110-118° C. The reaction mixture was maintained at reflux for 18-20 hrs and was analyzed by HPLC (to check for absence of compound IV). The reaction mixture was cooled at 25° C. to 30° C., filtered and washed with n-butanol and the mother liquor treated with 13 gm of fumaric acid (0.51 moles) to afford 2-(2-(4-dibenzo[b,f]-[1,4]thiazepine-1′-yl-1-piperazinyl)ethoxy)ethanol salt of fumaric acid which was recrystallized from 1430 cc ethanol. Yield-60-65 grams. Purity of 2-(2-(4-dibenzo[b,f]-[1,4]thiazepine-11-yl-1-piperazinyl)ethoxy)ethanol salt of fumaric acid was 99.5% (area % by HPLC).

### Example 16

### 2-(2-(4-dibenzo[b,f][1,4]thiazepine-11-yl-1-piper azinyl)ethoxy)ethanol salt of fumaric acid OR 11-[4-[2-(2-hydroxyethoxy)ethyl]-1-piperazinyl]dibenzo[b,f][1,4]thiazepine fumarate

A 1 liter round bottom flask equipped with stirring rod, thermo pocket, water condenser was charged with a solution of 11-piperazinyldibenzo[b,f][1,4]thiazepine [63 gm (0.22 moles)] in n-butanol 450 cc and the mixture was stirred for 15 min 25-30° C. To the resulting solution was added sodium carbonate [41.0 gm (0.39 moles)], tetra butyl ammonium bromide [16.0 gm (0.05 mole)[and 2-(2-chloroethoxy)ethanol [32.0 gm (0.257 moles)] at room temperature. The reaction mixture was heated to reflux at 110-118° C. The reaction mixture was maintained at reflux for 6-7 hrs and was analyzed by HPLC (to check for absence of compound IV). The reaction mixture was cooled to 25° C. to 30° C., filtered and washed with n-butanol and the mother liquor treated with 13 gm of fumaric acid ((0.51 moles) to afford 2-(2-(4-dibenzo[b,f][1,4] thiazepine-11-yl-1-piperazinyl)ethoxy)ethanol salt of fumaric acid which was recrystallized from 1275 cc ethanol. The wet material obtain was dried under vacuum at 50-55° C. to afford quetiapine fumarate. Dry weight of quetiapine fumarate is 60-65 gm. Purity of quetiapine fumarate was more than 99.5% (area % by HPLC).

### Example 17

### Example: Isolation of Impurity A & Impurity B

A 1 liter round bottom flask equipped with stirring rod, thermo pocket, nitrogen inlet, and water condenser were charged with 50 gm (0.22 moles) of dibenzo[b,f][1,4]thiazepine-11-(10H)one and 500 cc toluene and the mixture was stirred for 15 min. at room temperature. To the resulting solution was added phosphorus pentachloride 70 gm (0.33 moles) in five equal lots at 25° C. to 30° C. over 90 min. The reaction mixture was refluxed for 6 hrs at 110° C. The reaction mixture was analyzed by HPLC. The analysis showed that less than 2% of dibenzo[b,f][1,4]thiazepine(10)One was present. The reaction mixture was cooled to 20° C. to 25° C. for 30 min. The reaction mixture was dumped in pre chilled DM water (500 cc) at 10-15° C. and stirred for 30 min at 25-30° C. The layers were separated and the organic layer washed with saturated brine solution. The organic layer was distilled off under vacuum below 50° C. To the residue was added n-hexane and to remove traces of toluene. To the obtained oil was added 250 ml n-hexane and heated to 55-60° C. The reaction mixture was cooled to 20-25° C. and stirred for 45-60 min. The mixture was filtered and washed with n-hexane (50 ml). The obtained mother liquor was distilled-off under vacuum at below 50° C. To this mixture was added n-hexane at 50° C. and cooled to 20-25° C. The mixture was stirred for 45-60 min and filtered. The wet cake was washed with n-hexane. The obtained mother liquor was distilled off under vacuum at below 50° C.

The obtained residue contains enriched quantity of impurity A and impurity B. The impurity A and impurity B were isolated from residue using preparative TLC (Mobile phase: 0.50% ethyl acetate in toluene) or using chromatotron (mobile phase: n-hexane).

### Impurity A

7.57-7.60 (d, 2H), 7.40-7.43 (d, 1H), 7.32-7.34 (d, 1H), 7.04-7.25 (m, 7H), 6.90-6.95 (t, 1H), 1.16 (s, 3H).

M/Z=302 [M+H]

### Impurity B

7.52-7.54 (d, 1H), 7.35-7.40 (t, 2H), 7.25-7.28 (t, 1H), 7.20-7.21 (m, 4H), 6.97-7.09 (m, 3H), 6.85-6.88 (d, 1H), 1.16 (s, 3H).

M/Z=302 [M+H]

Having thus described the invention with reference to particular preferred embodiments and illustrative examples, those in the art may appreciate modifications to the invention as described and illustrated that do not depart from the spirit and scope of the invention as disclosed in the specification. The Examples are set forth to aid in understanding the invention but are not intended to, and should not be construed to, limit its scope in any way.

What is claimed is:

1. Isolated compound of the following structure:

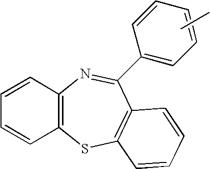

2. The compound of claim 1, wherein the compound has the following structure:

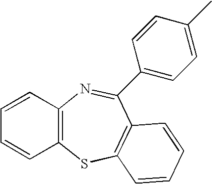

3. The compound of claim 1, wherein the compound has the following structure:

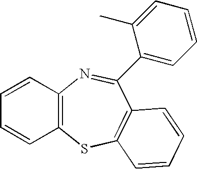

4. The compound of claim 1, wherein the compound is substantially free of a compound having the following structure: 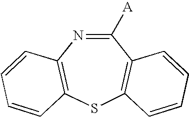
wherein A is a chlorine, bromine or iodine.

5. The compound of claim 4, wherein the halogen is chlorine.

6. A method for determining amount of an impurity or identifying such impurity forming as a result of chlorination of a compound having the following structure: 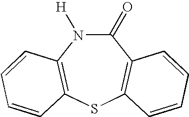
comprising carrying out chromatography on the product of the chlorination reaction, wherein compound of claim 1 is used as a reference standard for identifying or quantifying impurities.

7. A method for removing an impurity of the following structure: 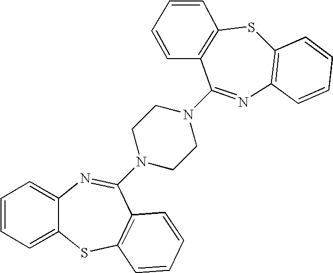
from 11-piperazinyldibenzo[b,f]thiazepine of formula [IV] comprising washing the 11-piperazinyldibenzo[b,f]thiazepine with an organic acid, or carrying out a slurry or crystallization from a C₁-C₅ alcohol.

8. The method of claim 7, wherein the organic acid is a C₁-C₈ acid aliphatic acid.

9. The method of claim 8, wherein the aliphatic acid is formic acid, acetic acid or adipic acid.

10. The method of claim 7, wherein a slurry is carried out.

11. The method of claim 7, wherein a crystallization is carried out.

12. The method of claim 7, wherein the alcohol is ethanol.

13. A process for preparing quetiapine comprising
a) reacting a compound III of Formula 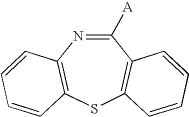
where A is chlorine, iodine or bromine, with piperizine to obtain a mixture of compound IV of formula: 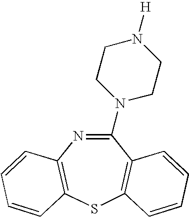
and an impurity of following structure: 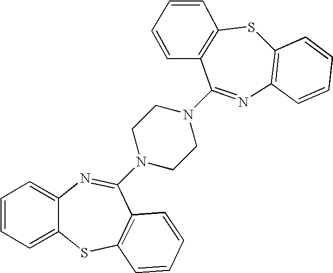
b) separating the impurity from compound of Formula IV by washing with an organic acid; and
c) converting compound of IV to quetiapine or a pharmaceutically acceptable salt.

14. A process for preparing a compound III of the following structure: 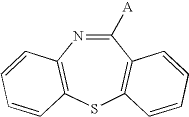
wherein A is chlorine, iodine or bromine, comprising combining a compound II of the following structure: 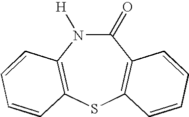
with a halogenating agent and an aliphatic halogenated hydrocarbon in the absence of a base to obtain compound III.

15. The process of claim 14, wherein the halogenated hydrocarbon is a C₁-C₈ hydrocarbon.

16. The process of claim 15, wherein the halogenated hydrocarbon is dichloromethane (DCM) or ethylene dichloride (EDC).

17. The process of claim 16, wherein the halogenated hydrocarbon is dichloromethane.

18. The process of claim 16, wherein the halogenated hydrocarbon is ethylene dichloride (EDC).

19. The process of claim 14, wherein A is chlorine.

20. The process of claim 14, wherein the halogenating agent is phosphorus pentachloride (PCl₅), phosphorous oxychloride, thionyl chloride or oxalylchloride.

21. The process of claim 20, wherein the halogenating agent is phosphorus pentachloride (PCl₅).

22. The process of claim 14, wherein molar ratio of the halogenating agent to compound II is of about 1.2 to about 1.6.

23. The process of claim 14, wherein temperature during reaction is about −5° C. to about −25° C.

24. The process of claim 14, wherein the compound III obtained has a purity of about 95% as area percentage HPLC.

25. The process of claim 24, wherein the compound III obtained has a purity of about 99% HPLC purity.

26. A process for preparing quetiapine or a pharmaceutically acceptable salt thereof comprising converting the compound prepared by the process of claim 14 to quetiapine or a pharmaceutically acceptable salt thereof.

27. A process for preparing quetiapine comprising the steps of:
a) halogenting a compound II of formula: 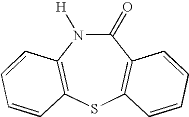
in the absence of a base by combining the compound II with an aliphatic halogenated hydrocarbon and a halogenating agent to obtain a compound III of formula: 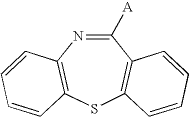
wherein A is chlorine, iodine or bromine;
b) reacting compound III with piperizine to obtain a compound IV of formula: 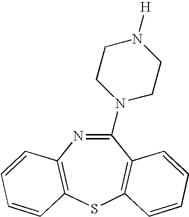
in a mixture with in impurity with following structure: 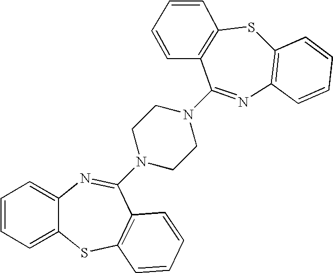
c) separating the from compound IV by at least one of combining the mixture with an organic acid or slurrying the mixture in a C₁-C₅ alcohol;
d) reacting compound IV with compound having the structure: 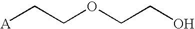
wherein A is chlorine, iodine or bromine, to obtain quetiapine of the following structure:

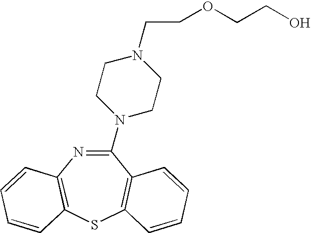

28. The process of claim 27, wherein the halogen is chlorine.

29. A process for reducing impurities present in the compound IV of formula: 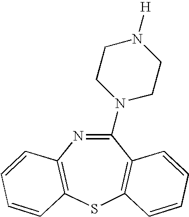
comprising reacting the compound with HCl to obtain a HCl salt of compound IV.

30. The process of claim 29, wherein the HCl salt is a 2HCl salt.

31. A process for reducing impurities present in the compound I of formula: 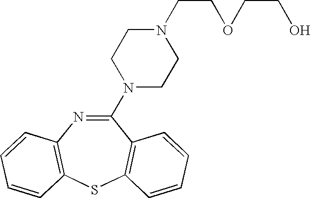
comprising reacting the compound with HCl to obtain a HCl salt of compound I.
